(19) United States
(12) Patent Application Publication
(10) Pub. No.: US 2010/0056894 A1
(43) Pub. Date: Mar. 4, 2010

(54) Self-Cleaning Membrane for Implantable Biosensors

(76) Inventors: Gerard L. Cotè, College Station, TX (US); Rebecca M. Gant, College Station, TX (US); Melissa A. Grunlan, College Station, TX (US); Yaping Hou, College Station, TX (US)

Correspondence Address: King & Spalding LLP, 401 Congress Avenue, Suite 3200, Austin, TX, 78701, US

(21) Appl. No.: 12/434,550

(22) Filed: May 1, 2009

Related U.S. Application Data
(60) Provisional application No. 61/050,491, filed on May 5, 2008.

(51) Int. Cl. A61B 5/145; G01N 27/26; C08L 83/04; B08B 7/00

(52) U.S. Cl. 600/365; 204/403.01; 525/100; 134/19; 600/309

(57) ABSTRACT

The present disclosure relates, according to some embodiments, to compositions, devices, systems, and methods including and/or for preparing and/or using a thermoresponsive nanocomposite hydrogel. In some embodiments, the disclosure relates to methods of preparing a hydrogel including, for example, photochemically curing an aqueous solution of NIPAAm and copoly(dimethylsiloxane/methylvinylsiloxane) colloidal nanoparticles (˜219 nm). At temperatures above a volume phase transition temperature (VPTT) of ˜33-34° C., a hydrogel may deswell and become hydrophobic, while lowering the temperature below a VPTT may cause the hydrogel to swell and become hydrophilic.

### CROSS-REFERENCE TO RELATED APPLICATION

This application claims the benefit of U.S. Provisional Application Ser. No. 61/050,491, filed May 5, 2008, entitled “A self-cleaning membrane for implantable biosensors,” the entire contents of which are incorporated herein by reference.

### FIELD OF THE DISCLOSURE

The present disclosure relates, in some embodiments, to compositions, devices, systems, and methods including and/or for preparing and/or using a thermoresponsive nanocomposite hydrogel.

### BACKGROUND OF THE DISCLOSURE

Implantable devices may be used for a wide variety of applications, including, for example, medical diagnostics and treatment. Optically based sensors may be desirable because after an initial implantation, they have the potential for continuous, non-invasive detection of the analyte of interest. In order for such an implant to function properly, the host response must be limited so as to not impair either analyte diffusion or signal propagation.

Even biocompatible materials that have no toxic effect on surrounding tissues elicit a host response. After an initial inflammatory response, specialized cells such as macrophages, monocytes, and lymphocytes attack the foreign body. If there is no significant biodegradation, fibroblasts will begin to form a tissue capsule on the surface of the implant one or two weeks after the number of macrophages decreases. Fibrous encapsulation may be influenced by many factors, including, for example, structure and morphology of the material, size of the implant, implantation site, and material biodegradability. The capsule may range in size from about 0.05 to 1.5 mm. This may be problematic because, for example, diffusion of the target analyte to the sensor may be limited. In some cases, a sensor may need to be calibrated to account for limited analyte diffusion, while in others, the non-uniformity of diffusion may defy calibration such that detected analyte may not be reflective of the systemic analyte concentration. In addition, the optical signal would be reduced by the additional light scatter and absorption, and local analyte levels could be altered by the surrounding cells.

### SUMMARY

Accordingly, a need has arisen for improved methods and compositions for preparing a biosensor membrane.

The present disclosure relates, in some embodiments, to compositions, devices, systems, and methods including and/or for preparing and/or using a thermoresponsive nanocomposite hydrogel. In some embodiments, the present disclosure relates to a thermoresponsive nanocomposite hydrogel that may be used to prepare a mechanically robust self-cleaning sensor membrane for an implantable biosensor. The present disclosure relates to methods and compositions for preparing biosensor membranes, according to some embodiments. In addition, the present disclosure relates to devices and systems that include a biosensor. A biosensor (e.g., an implantable biosensor) may comprise a membrane (e.g., a self-cleaning membrane) comprising, for example, a nanocomposite hydrogel. A biosensor may be configured and arranged, according to some embodiments, to detect one or more analytes including cholesterol, a cardiac marker, glucose, and combinations thereof A biosensor may comprise an implantable piece (e.g., a catheter).

In some embodiments, a nanocomposite hydrogel may be thermoresponsive and may display a temperature-modulated swelling and deswelling behavior, which may be cycled with a heating and/or cooling unit (e.g., an external unit) to control cellular, protein, and extracellular matrix adhesion in vivo. A nanocomposite hydrogel may have desirable mechanical strength, for example, more mechanical strength than a corresponding hydrogel without nanoparticles. A device and/or system may include a thermocycler configured and arranged to cycle the temperature of the nanocomposite hydrogel and/or surrounding area above and below the volume phase transition temperature (VPTT) of the nanocomposite hydrogel. A thermocycler may include a heating element and/or a cooling element. In some embodiments, a device and/or system may include a thermal controller configured and arranged to regulate the temperature of a hydrogel membrane and/or nearby (e.g., surrounding) material. A device and/or system may be scaled to any suitable size including, for example, a portable size.

According to some embodiments, methods and/or products may improve the lifetime of an implanted biosensor, for example, through the use of a nanocomposite hydrogel and an activation device. In some embodiments the activation mechanism and nanocomposite hydrogel may be configured to provide swelling and deswelling behavior using light, electrical field, pH, and/or other stimuli. A method of detecting an analyte (e.g., glucose) may comprise, according to some embodiments, providing a sensor system comprising a biosensor encased (at least partially) in a nanocomposite hydrogel and a thermocycler, implanting the biosensor in a subject, and cycling the temperature with the heating device around the volume phase transition temperature (VPTT). In some embodiments, a method of detecting an analyte may be practiced such that a subject's immune response and subsequent fibrous tissue encapsulation may be minimized or eliminated.

The disclosure also relates, in some embodiments, to a biosensor system comprising a photodiode and a biosensor membrane comprising a hydrogel, wherein the hydrogel may comprise one or more polysiloxane colloidal nanoparticles, cross-linked poly(N-isopropylacrylamide) and a hydrophobic and/or hydrophilic comonomer. In some embodiments, a method for cleaning an implanted biosensor membrane may comprise (a) providing an implanted biosensor membrane, wherein the biosensor membrane may comprise one or more polysiloxane colloidal nanoparticles, cross-linked poly(N-isopropylacrylamide) and a hydrophobic and/or hydrophilic comonomer, (b) providing a thermocycler in thermal communication with the biosensor membrane; and (c) cycling the temperature of the thermocycler around the volume phase transition temperature (VPTT) of the biosensor membrane. According to some embodiments, a method for detecting an analyte may comprise (a) providing an implanted biosensor configured and arranged to detect an analyte, the biosensor comprising a biosensor membrane, wherein the biosensor membrane may comprise one or more polysiloxane colloidal nanoparticles, cross-linked poly(N-isopropylacrylamide) and a hydrophobic and/or hydrophilic comonomer, and (b) detecting the analyte.

### BRIEF DESCRIPTION OF THE DRAWINGS

Some embodiments of the disclosure may be understood by referring, in part, to the present disclosure and the accompanying drawings, wherein:

FIG. 1 illustrates a wrist-band type sensor system according to a specific example embodiment of the disclosure;

FIG. 2 illustrates thermocycling stages for a self-cleaning sensor membrane according to a specific example embodiment of the disclosure;

FIG. 3 illustrates a competitive binding assay according to a specific example embodiment of the disclosure;

FIG. 4A illustrates a hydrogel in a swollen, hydrophilic state below the VPTT with attached acclimated cells according to a specific example embodiment of the disclosure;

FIG. 4B illustrates a hydrogel in a dehydrated, hydrophobic state above the VPTT with acclimated cells attached according to a specific example embodiment of the disclosure;

FIG. 5 illustrates volume equilibrium swelling ratios as a function of temperature for (A) pure poly(N-isopropylacrylamide)(PNIPAAm), (B) PNIPAAm+nanoparticles, (C) porated PNIPAAm+nanoparticles, and (D) 60% PEG according to a specific example embodiment of the disclosure;

FIG. 6 is a phase transition time plot illustrating material swelling upon cooling to 25° C., followed by de-swelling when the temperature was raised to 37° C. for (A) pure PNIPAAm, (B) PNIPAAm+nanoparticles, and (C) porated PNIPAAm+nanoparticles according to a specific example embodiment of the disclosure;

FIG. 7 illustrates a side-by-side cell setup for measuring diffusion through the polymer materials according to a specific example embodiment of the disclosure;

FIG. 8 illustrates a comparison of attached cells according to a specific example embodiment of the disclosure: (A) Modified PNIPAAm/nanoparticle hydrogel at initial 37° C. (B) Polystyrene control plate at initial 37° C. (C) Modified PNIPAAm/nanoparticle hydrogel after the temperature was lowered to 25° C. (D) Control at 25° C.;

FIG. 9 illustrates detachment of cells in response to temperature variation for (A) pure PNIPAAm, (B) PNIPAAm+nanoparticles, (C) porated PNIPAAm+nanoparticles, (D) 60% PEG, and (E) polystyrene control plate according to a specific example embodiment of the disclosure.

FIG. 10 illustrates DSC thermograms for nanocomposite hydrogels (b-e) and pure PNIPAAm hydrogel (a);

FIG. 11 illustrates a storage modulous (G′) of nanocomposite hydrogels (b-e) and pure PNIPAAm hydrogel (a) measured in the compression mode; and

FIG. 12 illustrates crosslinked polysiloxane nanoparticles according to example embodiments of the disclosure.

### DETAILED DESCRIPTION

Fibrous tissue encapsulation may slow the diffusion of the target analyte to an implanted sensor and compromise the optical signal. Poly(N-isopropylacrylamide) (PNIPAAm) hydrogels are thermoresponsive, exhibiting temperature-modulated swelling behavior that may be used to prevent biofouling. PNIPAAm hydrogels may not have desired mechanical strength. The present disclosure relates, according to some embodiments, to a thermoresponsive nanocomposite hydrogel that may be used to prepare a mechanically robust self-cleaning sensor membrane for an implantable biosensor. In some embodiments, the disclosure further relates to methods of preparing a hydrogel including photochemically curing an aqueous solution of NIPAAm and copoly(dimethylsiloxane/methylvinylsiloxane) colloidal nanoparticles (˜219 nm). At temperatures above the volume phase transition temperature (VPTT) of ˜33-34° C., the hydrogel may deswell and become hydrophobic, while lowering the temperature below the VPTT may cause the hydrogel to swell and become hydrophilic. The ability of an example embodiment of this material to minimize biofouling via temperature-modulation while maintaining sensor viability has been tested using glucose as a target analyte. PNIPAAm composite hydrogels with and without poration were compared to a pure PNIPAAm hydrogel and a non-thermoresponsive PEG hydrogel. In accordance with observed results, poration may lead to a substantial increase in diffusion. In addition, cycling the temperature of the nanocomposite hydrogels around the VPTT may cause significant detachment of GFP-H2B 3T3 fibroblast cells.

The present disclosure relates, in some embodiments, to methods, compositions, devices, and/or systems for detecting an analyte. A biosensor, according to some embodiments, may have long term stability and/or functionality permitting it to be used in vivo successfully. In some embodiments, a biosensor may be configured and arranged to reduce and/or minimize the host response along with potential fibrous encapsulation. A biosensor (e.g., an implantable biosensor) may comprise a membrane (e.g., a self-cleaning membrane) comprising a thermoresponsive PNIPAAm nanocomposite hydrogel according to some embodiments. A thermoresponsive PNIPAAm nanocomposite hydrogel may have, in some embodiments, a volume phase transition just below body temperature (e.g., at about 33-34° C.). According to some embodiments, diffusion of an analyte (e.g., glucose) may be similar to a PEG hydrogel below the VPTT, but significantly decreased at temperatures above the VPTT. Poration of a nanocomposite hydrogel may improve (e.g. dramatically improve) analyte diffusion (e.g., glucose diffusion) above the VPTT in some embodiments. A nanocomposite hydrogel, according to some embodiments, may release up to about 10-40%, up to about 40-70%, up to about 70-80% of the initially attached fibroblast cells when the temperature is decreased below the VPTT. In some embodiments, a nanocomposite hydrogel may experience little or no significant reattachment during subsequent heating and cooling cycles. Illustrative results obtained with example embodiments show that a nanocomposite PNIPAAm hydrogel may be useful as a self-cleaning membrane for implantable biosensors. A thermoresponsive nanocomposite hydrogel, according to some embodiments, may reduce and/or prevent capsule formation.

Without being limited to any particular mechanism of action “smart” materials, in some embodiments, may reversibly switch from a hydrophilic to hydrophobic state in aqueous media in response to an external stimulus. Thermoresponsive hydrogels may be crosslinked, three dimensional polymer networks that reversibly swell with and then expel aqueous media in response to temperature changes. Thermoresponsive hydrogels may be prepared by crosslinking polymers which exhibit a lower critical solubility temperature (LCST). Examples include poly(N-isopropylacrylamide) (PNIPAAm) (LCST, ˜32° C.), which is soluble in water below the LCST and reversibly insoluble above the LCST. Crosslinked PNIPAAm hydrogels undergo a reversible volume phase transition in water from a swollen state to a deswollen state above their volume phase transition temperature (VPTT; ˜33-34° C.).

Without being limited to any particular mechanism of action, thermosensitive polymers, according to some embodiments, may be soluble in water at temperatures below the volume phase transition temperature (VPTT) due to hydrogen bonding with water molecules. Above the VPTT, the hydrogen bonding may be disrupted and become insufficient to maintain the macromolecule in solution resulting in its collapse. Basically, the hydrophobic interactions between the polymer molecules may become more favorable than the interactions between the polymer and water. Thermosensitive materials may be used in a variety of applications including drug delivery, gene delivery, and cell detachment. Stimuli-responsive polymers may also be used for culturing cells that are too sensitive to be enzymatically detached from a culture dish. In some embodiments, cells may be cultured on a thermoresponsive surface and later released as an intact sheet of cells. A thermoresponsive hydrogel, in some embodiments, may be desirable for in vivo use because it may exhibit a sharp change in hydration over a short range of temperature. Additionally, the high water content of the hydrogels may make them relatively flexible, minimizing the local tissue irritation that may occur with a stiffer material. However, a highly hydrated state of the polymers may contribute to poor mechanical integrity.

According to some embodiments, a self-cleaning nanocomposite hydrogel may display temperature-dependent swelling behavior that affects cellular adhesion. Thermoresponsive poly(N-isopropylacrylamide) (PNIPAAm) hydrogels may be biocompatible and may have a VPTT just below body temperature (˜33-34° C.) in some embodiments. A PNIPAAm hydrogel in a highly swollen state may have undesirably low mechanical strength. Mechanical strength may be increased by copolymerization with another polymer, but this may lead to a change in transition temperature, which may undesirably impact its in vivo utility. Nanocomposite hydrogels prepared by introducing polysiloxane colloidal nanoparticles into PNIPAAm hydrogel matrices may exhibit an increase in mechanical strength without significantly altering the phase transition temperature. In some embodiments, a nanocomposite hydrogel may be configured and arranged with or without poration.

Nanoparticles, in some embodiments, may be prepared according to Hou Y et al,. “Thermoresponsive nanocomposite hydrogels with cell-releasing behavior,” POLY Preprints (Amer. Chem. Soc., Div. Poly. Mater. Sci. Eng.), 2009, 50 (1), 21-22 and/or “Thermoresponsive nanocomposite hydrogels with cell-releasing behavior,” Hou, Y.; Burkes, J. C.; Lee, S. D.; Bullick, A. S.; Hahn, M. S.; Grunlan, M. A. presented at the 237th ACS National Meeting, Salt Lake City, Utah, United States, Mar. 22-25, 2009 [talk], the entire contents of which are hereby incorporated by reference.

In some embodiments, increasing levels of nanoparticles may lead to improved mechanical properties. For example, tensile modulus, ultimate tensile strength, and % strain at break and compression modulus may increase with nanoparticle content. Nanoparticles may have an average diameter of from about 1 to about 25 nm, from about 25 nm to about 50 nm, less than about 50 nm, from about 50 nm to about 220 nm, and/or more than about 220 nm.

In some embodiments, a biosensor (e.g., an implantable biosensor) may comprise one or more agents (e.g., nitric oxide and/or other drugs) that reduce, block, and/or prevent a fibrous tissue response. These agents may not be desirable in some circumstances. For example, such agents may not be desirable for long-term use.

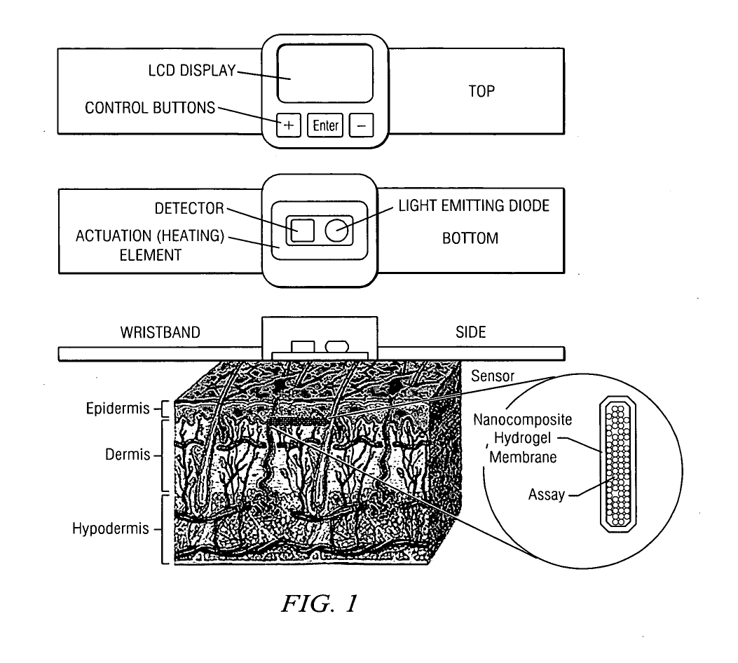

A biosensor system may be configured and arranged as a wrist-band as illustrated in FIG. 1. A wrist-band sensor system may include an upper or outer surface comprising display and integrated (e.g., touchscreen) or separate controls (e.g., buttons). A wrist-band sensor system may include a lower or inner surface comprising, for example, a light-emitting diode, a detector (e.g., an implantable biosensor), and an actuation element (e.g., a heating element, a cooling element, and/or a thermocycler). The bottom surface may be configured and arranged to face and/or contact a subject (e.g., a subject's wrist).

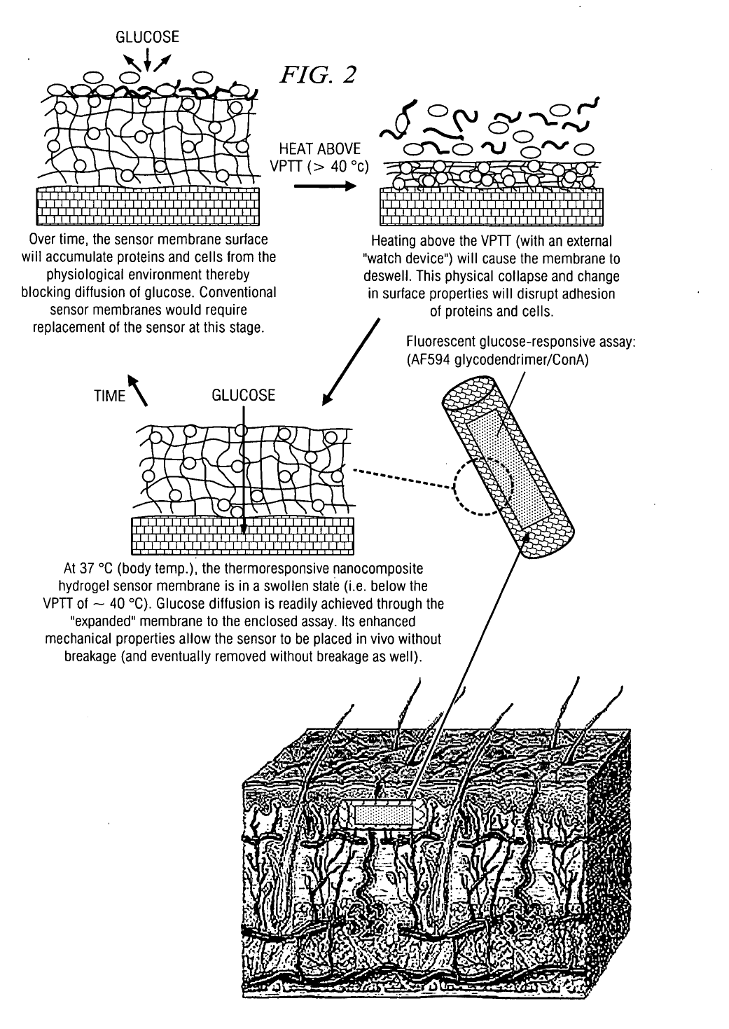

FIG. 2 illustrates an example embodiment of events that may occur during a thermal cycle of a self-cleaning sensor membrane. Over time, a sensor membrane surface may accumulate proteins and cells from the physiological environment thereby blocking diffusion of glucose as shown on the upper left. Conventional sensor membranes may require the replacement of the sensor at this stage. Heating a sensor membrane material above its VPTT (with an external “watch device”) may cause it to Deswell. This physical collapse and change in surface properties may disrupt adhesion of proteins and cells as shown in the upper right of FIG. 2. At 37° C. (body temperature) a thermoresponsive nanocomposite hydrogel sensor membrane may be in a swollen state (i.e., below the VPTT of ˜40° C.). Glucose diffusion may be achieved (e.g., readily achieved) through the “expanded” membrane to the enclosed assay as shown in the center below. Its enhanced mechanical properties may allow a sensor to be placed in vivo without breakage (and eventually removed without breakage as well) as shown on the far right.

A self-cleaning sensor membrane may be prepared with a thermoresponsive nanocomposite hydrogel material (e.g., a PNIPAAm-co-AA hydrogel matrix and polysiloxane nanoparticles) and enclose a fluorescent glucose-responsive assay.

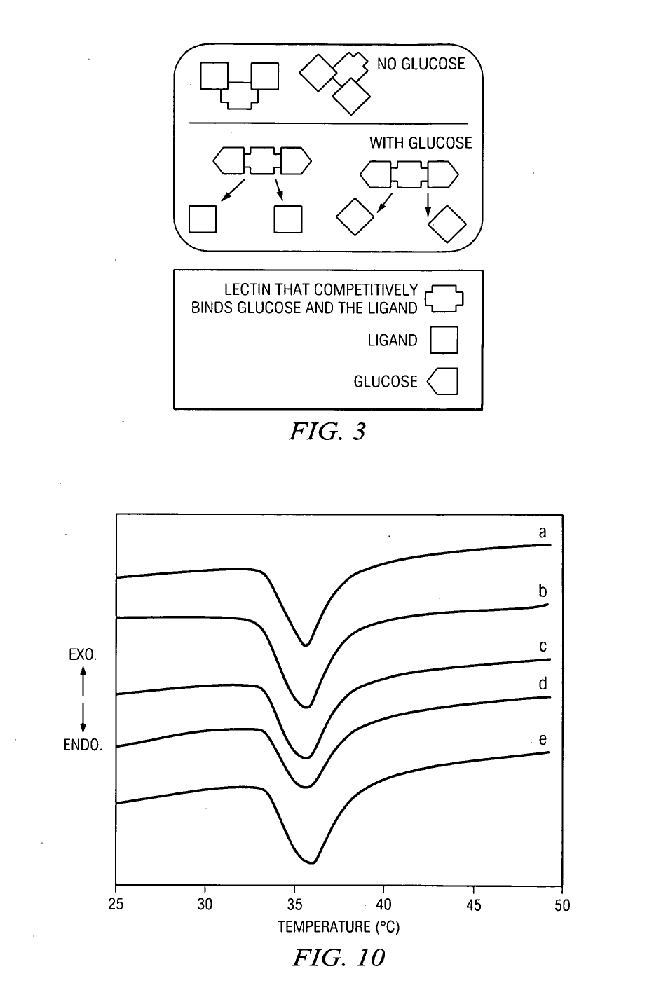

In some embodiments, a competitive binding assay for glucose may be used within one or more polymer biospheres as shown in FIG. 3.

A comonomer, according to some embodiments, may be desirable to set and/or adjust the VPTT to a precise value. For instance, the VPTT of PNIPAAm-based hydrogels may be decreased by incorporation of hydrophobic comonomers or increased with hydrophilic comonomers. Hydrogels may be formed from curing aqueous (or non-aqueous) precursor solutions comprised of (1) NIPAAm monomer and optionally comonomer(s) or (2) PNIPAAm polymer and optionally copolymers of PNIPAAm (e.g. copoly(N-isopropylacrylamide-acrylic acid) (NIPPAm-co-AA)) or some combination thereof. A crosslinker such as NN′-methylenebisacrylamide may or may not be included to increase crosslinking. Crosslinking may be used to accelerated with thermal, redox, UV, or other catalysts along with heat or UV-light.

As will be understood by those skilled in the art who have the benefit of the instant disclosure, other equivalent or alternative compositions, devices, methods, and systems for preparing a biosensor membrane, using (e.g., cleaning) a biosensor membrane, and/or assaying an analyte can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of biosensor membranes may be varied. In some embodiments, biosensor membranes, monomers, polymers, hydrogels, nanoparticles, thermocyclers and/or detectors (e.g., photodiodes) may be interchangeable. Interchageability may allow, for example, VPTT to be custom adjusted (e.g., by adjusting the hydrophobicity/hydrphilicity). In addition, the size of a device and/or system may be scaled up (e.g., to be used for adult subjects and/or macroanalysis) or down (e.g., to be used for juvenile subjects and/or micro or nanoscale analysis) to suit the needs and/or desires of a practitioner. Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, a range of endpoint of about 50 may one the one hand include 50.5, but not 52.5 or 55 in the context of a range of about 5 to about 50 and, on the other hand, include 55, but not 60 or 75 in the context of a range of about 0.5 to about 50. In addition, it may be desirable in some embodiments to mix and match range endpoints. Also, in some embodiments, each figure disclosed (e.g., in one or more of the Examples and/or Drawings) may form the basis of a range (e.g.,+/− about 10%, +/− about 100%) and/or a range endpoint. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

All or a portion of a device and/or system for preparing a biosensor membrane, using (e.g., cleaning) a biosensor membrane, and/or assaying an analyte may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### Examples

Some specific example embodiments of the disclosure may be illustrated by one or more of the examples provided herein.

### Example 1

### Materials

Liquid poly(ethylene glycol) diacrylate (MW 575), poly(ethylene glycol) (MW 1000), N-isopropylacrylamide (NIPAAm), potassium persulfate, sodium carbonate, and glucose (HK) assay were purchased from Sigma-Aldrich (St. Louis, Mo.). 2-Hydroxy-2-methyl-1-phenyl-1-propanone (Darocur 1173) and 1-[(2-hydroxyethoxy)-phenyl]-2-hydroxy-2-methyl-1-propane-1-one (Irgacure 2959) were obtained from Ciba Specialty Chemicals (Tarrytown, N.Y.). N,N′-methylene bis-acrylamide (BIS) was purchased from Acros Organics (Geel, Belgium). Octamethylcyclotetrasiloxane (D₄) and 1,3,5,7-tetramethyl-1,3,5,7-tetravinylcyclotetrasiloxane (D₄^{Vi}) came from Gelest and dodecylbenzenesulfonic acid (DBSA, BIO-SOFT®) came from Stepan. The Slide-A-Lyzer dialysis cassette (MWCO 10,000) was obtained from Pierce (Rockford, Ill.). All aqueous experiments were performed with deionized water with a resistance of 18 MΩ·cm (Millipore, Billerica, Mass.).

### Example 2

### Nanoparticle Synthesis

Crosslinked polysiloxane colloidal nanoparticles with an average diameter of 219 nm and particle sizes ranging from 106 to 531 nm were prepared according to Hou et al. First, cationic emulsion polymerization of octamethylcyclotetrasiloxane and 1,3,5,7-tetramethyl-1,3,5,7-tetravinylcyclotetrasiloxane with dodecylbenzenesulfonic acid as an initiator and surfactant produced uncrosslinked nanoparticles. After adjusting the pH to 7 with Na₂CO₃, the nanoparticles were crosslinked by free radical reaction of the vinyl groups of the copoly(dimethylsiloxane/methylvinylsiloxane) inside the particles. The nanoparticles were purified using a dialysis cassette with a molecular weight cut off (MWCO) of 10,000 with daily water changes for 3 days.

### Example 3

### Sensor Membrane Fabrication

Three PNIPAAm-based hydrogels were prepared along with a poly(ethylene glycol) (PEG) hydrogel as a control. The composition of each precursor solution is shown in Table 1. (A) PNIPAAm aqueous solution was composed of 12.5 wt % NIPAAm monomer, 2 wt % BIS crosslinker, and 1 wt % Irgacure-2959 photoinitiator. (B) The modified PNIPAAm nanocomposite solution included 1 wt % polysiloxane colloidal nanoparticles (based on total precursor solution weight) in addition to the above materials. The solutions were purged with nitrogen for 10 minutes and injected into a glass mold. The mold was sealed and submerged into an ice water bath (˜7° C.). The solutions were photopolymerized by exposure to UV light for 10 minutes (9 mW/cm², λₚₑₐₖ=365 nm, UVP UV-Transilluminator). The hydrogels were then rinsed with DI water and soaked for at least 24 hours to remove impurities and allow for adequate hydration. (C) Porated modified PNIPAAm nanocomposite hydrogels were created by adding 20% PEG (MW 1000) to the precursor solution, which was then removed by extended soaking for at least 72 hours in DI water after photopolymerization. (D) A poly(ethylene glycol) diacrylate solution was prepared as a non-thermoresponsive control. Aqueous PEG hydrogels were created with 60% v/v poly(ethylene glycol) diacrylate (PEG-DA) (MW 575) and 1% v/v Darocur 1173 photoinitiator. The PEG polymer precursor was cured at room temperature for 3 seconds.

### Example 4

### Measurement of Equilibrium Swelling

The swelling/deswelling behavior of each of the four material configurations (A-D) was studied by measuring the wet weight (w_{wet}) of each material. Polymer slabs (1 cm×1 cm×500 μm) were submerged in DI water of a specific temperature for 24 hours prior to measurement. The dry weight (w_{dry}) was determined by drying the polymer slabs in an oven (40° C.) for 24 hours before measurement. The swelling ratio is defined as:

$$S R = \frac{{w}_{wet} - {w}_{dry}}{{w}_{dry}}$$

### Example 5

### Measurement of Swelling/Deswelling Kinetics

The reaction time for the volume phase transition of each of the four materials (A-D) was studied by starting with hydrogels maintained at 37° C. and introducing them to 25° C. solution, taking wet weight measurements every five minutes for one hour. The solution was maintained at 25° C. for the following 24 hours to allow for accurate measurement of the fully transitioned material. The temperature was raised back to 37° C. and measurements were again made for one hour, followed by the measurement of the equilibrated hydrogels at 24 hours.

### Example 6

### Measurement of Glucose Diffusion Rates

A diffusion study was performed on each of the four hydrogels (A-D) using glucose as an example analyte. Each 500 μm thick hydrogel slab was tested in a side-bi-side diffusion cell (PermeGear, Bethlehem, Pa.) with glucose solution in the donor chamber and deionized water in the receptor chamber. The solutions were stirred to maintain constant concentrations and a water jacket maintained a consistent temperature through the system. Samples were taken every 10 minutes from the receptor chamber. The glucose concentration was measured using a glucose (HK) assay and the absorbance at 340 nm was determined with a spectrophotometer (USB 2000, Ocean Optics, Dunedin, Fla.).

### Example 7

### Measurement of Thermoresponsive Cell Release Characteristics

Each of the hydrogels (A-D), along with a polystyrene cell culture dish as a control, were UV sterilized and inoculated with GFP-H2B 3T3 mouse fibroblast cells. The cells were maintained in DMEM with 10% fetal calf serum and antibiotics at 37° C. and 5% CO₂. The inoculated hydrogels were incubated overnight. Images were taken every 20 minutes, beginning with 37° C. (Nikon Eclipse TE 2000-S). At the end of each hour, the temperature was varied between 37° C. and 25° C.

### Example 8

### Material Properties

Three PNIPAAm-based hydrogels (A-C) were prepared along with a poly(ethylene glycol) (PEG) hydrogel (D) as a control in order to assess the potential for use as a membrane for implantable biosensors. A pure PNIPAAm hydrogel (A) was used as a comparison for the properties of the nanocomposite hydrogel (B). The third PNIPAAm-based polymer (C) was developed in order to overcome the diffusion limits encountered at temperatures above the VPTT.

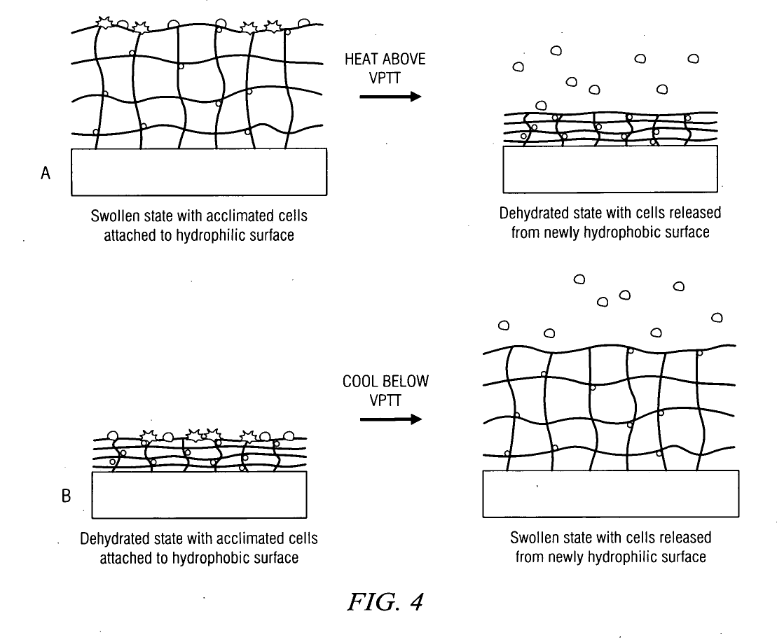

The PNIPAAm-based materials (A-C) are hydrophilic and in a hydrated, swollen state at room temperature. As the temperature is increased above the volume phase transition temperature (VPTT), the hydrogels become hydrophobic and exude water. Stretching forces from the swelling/deswelling behavior and/or changes in hydrophobicity may prevent proteins and cells from attaching to the material, as shown in FIG. 4. FIG. 4A shows the hydrogel in a swollen, hydrophilic state below the VPTT with attached acclimated cells. Above the VPTT, the hydrogel exudes water and the cells are released as the surface becomes hydrophobic. FIG. 4B shows the hydrogel in a dehydrated, hydrophobic state above the VPTT with acclimated cells attached. As the hydrogel is cooled, it swells and becomes more hydrophilic, inducing detachment of cells.

Pure PNIPAAm hydrogels (A) may be limited by the low strength of the gels when they are in a highly swollen state. Nanocomposite hydrogels (B) were prepared with polysiloxane colloidal nanoparticles in order to improve the mechanical properties. Higher nanoparticle concentrations led to increased mechanical strength, but lessened the optical transparency of the material. The nanocomposite hydrogels were prepared with 1 wt % nanoparticles because this concentration allowed sufficient light propagation for biosensing purposes. In addition, each of the PNIPAAm-based polymers (A-C) were photopolymerized at ˜7° C. because photopolymerization at temperatures below the LCST may produce PNIPAAm hydrogels with homogeneous morphology, resulting in optically clear hydrogels with improved mechanical integrity.

By controlling the local temperature, it may be possible to direct the cell detachment from an implanted device in vivo. Cycling the temperature around the VPTT may prevent any long term attachment and fibrous encapsulation, extending the useable lifetime of the sensor. To assess the potential of this unique hydrogel, three studies were performed: (1) a hydration study to examine the phase transition characteristics, (2) a diffusion study of the hydrogels using glucose as the analyte of interest, and (3) cell culture studies to assess cell detachment as a function of the hydrogel and temperature.

### Example 9

### Temperature-Dependent Equilibrium Swelling

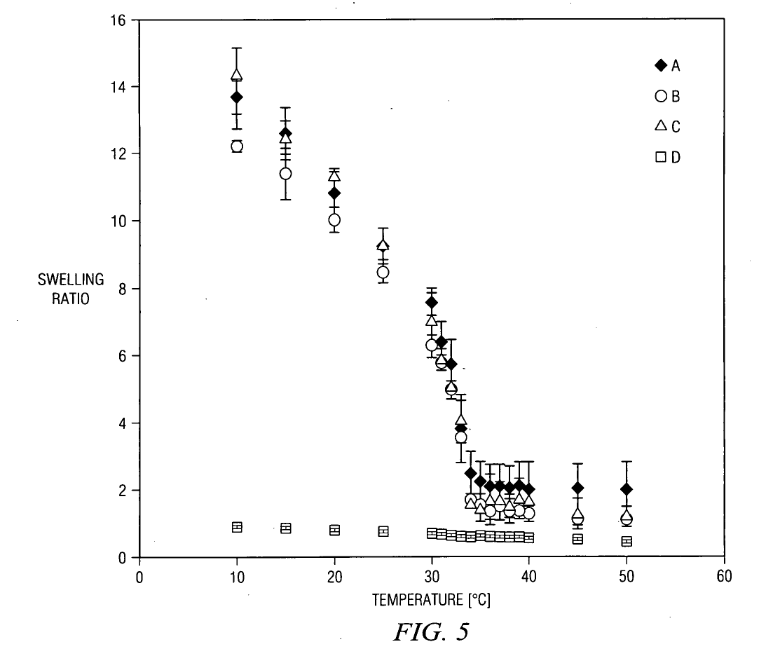

The temperature-dependent equilibrium swelling behavior of each of the four material configurations (A-D) was measured from 10 to 50° C. As shown in FIG. 5, PNIPAAm materials may absorb significantly more water at lower temperatures and become deswollen at temperatures above the VPTT. The PEG hydrogel (D) served as a control, maintaining a constant level of hydration over the entire thermal range. The VPTT does not change vs. that of pure PNIPAAm with addition of the polysiloxane nanoparticles as it would if NIPAAm was copolymerized with a second monomer.

### Example 10

### Swelling/Deswelling Kinetics]

The point at which the volume phase transition occurs is approximately 33-34° C., which may be particularly useful for biological applications. A biosensor of this material may be implanted just below the skin and the temperature could be periodically lowered using an external device, triggering the transition, and releasing any adhered cells.

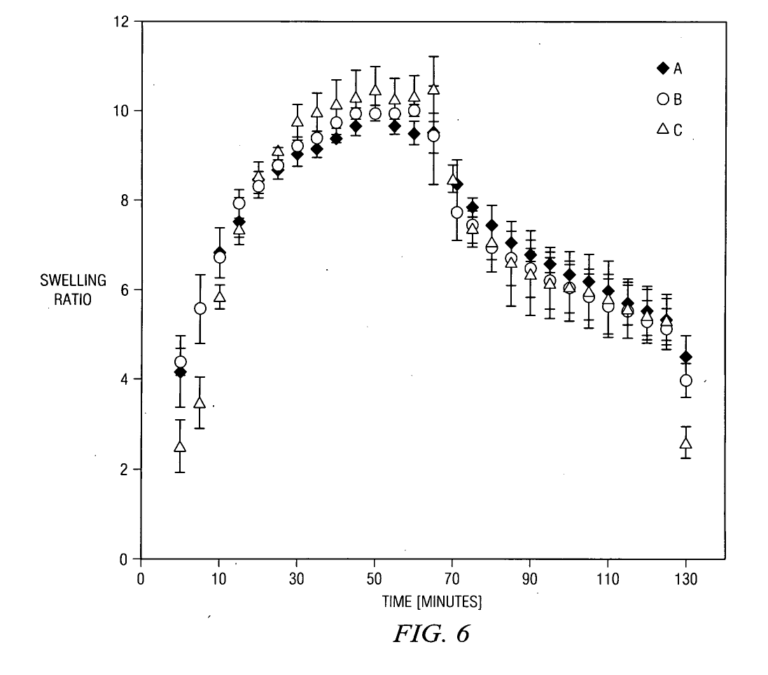

Swelling and deswelling kinetics were studied by measuring the transition time between 25 and 37° C. FIG. 6 shows that the polymers reach 90% hydration in approximately 25 to 30 minutes. After increasing the temperature, there is an initial rapid shrinking, followed by a slower deswelling. The water expulsion from the center of the hydrogels may be partially restricted by the immediate surface shrinking. The deswelling process takes longer than swelling, but the transition begins occurring immediately with no lag time. This information would be important for the design of a thermal cycling element to optimize the anti-fouling properties of an implanted sensor. The swelling and deswelling process is dependent on diffusion. Therefore the time of swelling is proportional to the square of the size of the material and the diffusion coefficient. In order to minimize swelling time, the smallest dimension, or thickness, could be minimized.

### Example 11

### Glucose Diffusion

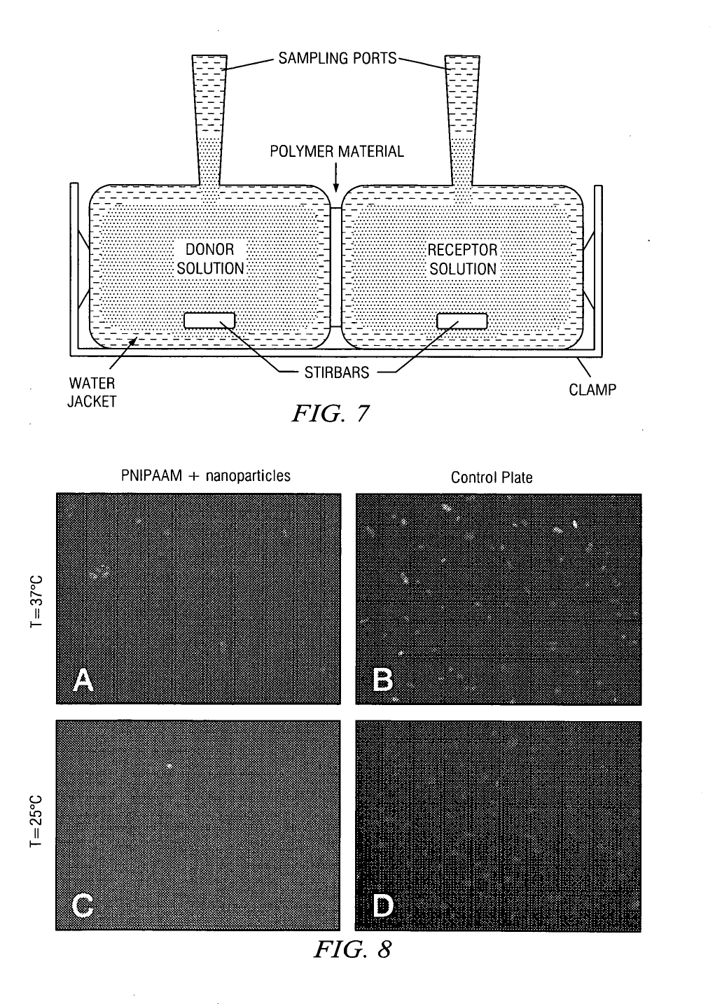

A diffusion study was performed to analyze the transport of small analytes through each material. Glucose was used as an example analyte as it is a molecule of high interest for implantable sensors. Each of the materials was tested in a side-by-side diffusion cell, as shown in FIG. 7.

In order to calculate the diffusion coefficients, analysis using Fick's second law of diffusion was performed.

$$\frac{\partial c}{\partial t} = D \frac{{\partial }^{2} c}{\partial {x}^{2}}$$

where c is the concentration in the membrane, t is time, D is the diffusion coefficient, and x is distance. This assumes that the chambers are well mixed and the component concentrations are the same at membrane surface and in the bulk fluid. This may be solved to:

$${Q}_{t} = \frac{{ADC}_{1}}{l} ( t - \frac{{l}^{2}}{6 D} )$$

where Qₜ is the total amount of solute transferred through the membrane until time t, A is membrane area, C₁ is the solute concentration in the donor chamber, and 1 is membrane thickness. The lag time method may be used to calculate the diffusion coefficient using the intercept of the linear part of the curve obtained by plotting Q vs. time. Since using the slope may lead to a much closer estimation of diffusivity, this was used for these calculations.

PEG-based polymers may not significantly limit the transport of glucose to an encapsulated sensor. So, a metric by which to assess the nanocomposite hydrogels (B, C) is whether they have similar glucose diffusion as the PEG hydrogel (D). As shown in Table 2, the diffusion rates in the pure PNIPAAm hydrogel (A) and nanocomposite hydrogel (B) decrease significantly when the temperature was raised to 37° C. due to the inherent material hydrophobicity and tight mesh size above the VPTT. To overcome this potential inconvenience, a porated nanocomposite hydrogel (C) was created. It showed a significant increase in the diffusion rate, particularly above the VPTT. Without being limited to any particular mechanism of action, the additional improvement may be due to the travel of glucose through the pores, as opposed to becoming entrapped within the collapsed hydrogel matrix.

### Example 12

### Cell Release

In order to determine if the swelling/deswelling behavior affects cellular adhesion, hydrogels (A-D) were sterilized and inoculated with GFP-H2B 3T3 mouse fibroblast cells. These cells were chosen to mimic an implant environment and because the GFP-labeled chromatin is easy to image. FIG. 8 shows fluorescence images of the cells attached to the materials. Brightfield validation showed the cell morphology with the fibroblasts attached to the material surfaces. While the polystyrene control plate remained covered in cells, the modified PNIPAAm material exhibited a significant decrease in the number of attached cells when the temperature was lowered below the VPTT.

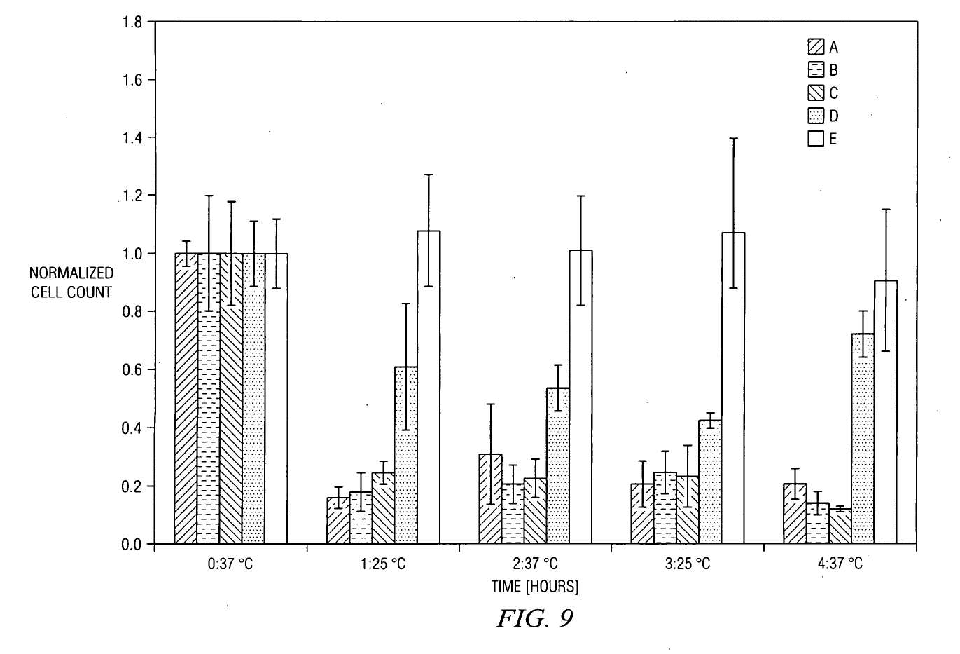

The relative number of cells attached throughout the thermal cycling process is shown in FIG. 9. The initial cell count for each material was used to normalize the following data. The polystyrene control remained relatively stable throughout the experiment, while thermoresponsive hydrogels (A-C) exhibited a significant decrease in number of attached cells after the initial temperature variation and never recovered. The PEG hydrogel (D) showed an initial decrease in the number of attached cells, likely due to its inherent anti-fouling properties. Although PEG-coated materials have been shown to resist cellular attachment for up to two weeks in vitro, it is less successful in vivo. After 4 weeks in subcutaneous mouse tissue, PEG-coated implants have been observed to experience fibrous encapsulation.

### Example 13

### Dynamic Mechanical Analysis (DMA) and Tensile Test

DMA of hydrogels was measured in the compression mode with a dynamic mechanical analyzer (TA Instruments Q800) equipped with parallel-plate compression clamp with a diameter of 40 mm (bottom) and 15 mm (top). Swollen hydrogel discs of constant dimension (13 mm diameter, 1.5 mm thickness) were punched from a hydrogel sheet and clamped between the parallel plates. Silicone oil was then placed around the exposed edges of the hydrogel to prevent dehydration. Following equilibration at the 25° C. (5 min), the samples were tested in a multi-frequency-strain mode (1-65 Hz) at the temperature of 25° C. (below the VPTT). Results reported are based on the average of five individual specimens.

Tensile tests of hydrogels ring specimens were measured on a TA Instruments DMA Q800 operating in the tension mode. Specimens with a ring geometry were prepared by cutting a portion from a hydrogel tube produced from the double wall tubular mold (ID ¼ 3 mm, OD ¼ 7.5 mm). Individual rings (w3 mm width) were cut from the central portion of the appropriate hydrogel tube using a clean razor blade and sample dimensions measured with an electronic caliper. Each hydrogel ring was blotted with filter paper and loaded onto custom aluminum bars gripped directly into DMA tension clamps so that the upper and lower bars were located inside the ring. Samples were subjected to a constant strain (1 mm/min) until they broke at the center of one side of the ring. Stress was calculated from the measured force divided by the cross-sectional area of two rectangles with sides equal to the width and wall thickness of the ring. The gauge length corresponded to the outer diameter of the ring minus the wall thickness. The following parameters were determined: (1) tensile modulus, (2) ultimate tensile strength (UTS), and (3) percent strain at break. The tensile modulus was obtained from the slope of the linear part of the stress-strain curve. The UTS represents the maximum stress prior to failure. Strain was calculated from the measured displacement divided by the gauge length. Results reported are the average result of three specimens cut from central portion of the same hydrogel tube.

The mechanical properties of biomaterials such as hydrogels directly affect their utility and performance. Because of their frequent use to measure hydrogel mechanical properties, both dynamic mechanical analysis (DMA) and tensile tests were utilized in this study. Mechanical properties of hydrogels in a swollen state are most relevant to their end-use applications. However, mechanical testing of swollen hydrogels is challenging because the difficulty in maintaining hydration and their high-water content/low-polymer mass to unit volume make them mechanically weak and difficult to handle. Thus, during DMA, silicone oil was placed around the hydrogel disc specimen sandwiched between two compression clamps to inhibit water loss. Tensile testing of flat, rectangular hydrogel specimens with ends secured in tension grips is often complicated by sample slippage from or breakage at the grip. Thus, specimens with a ring geometry were employed to minimize slippage/breakage for improved accuracy. Ring specimens also allowed their rapid mounting on tensile bars so that testing was completed before significant water loss occurred.

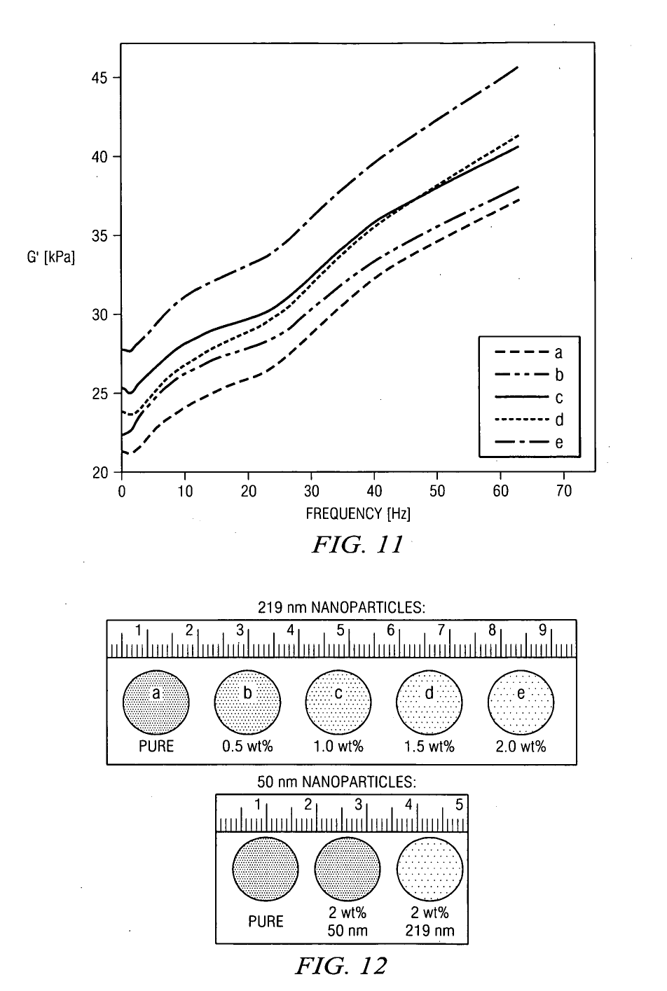

In DMA, the measured storage modulus (G′) is related to a materials stiffness or resistance to deformation. At all frequencies (1-65 Hz), the G′ of the nanocomposite hydrogels (b-e) was higher than that of the pure PNIPAAm hydrogel (a) and increased with higher levels of polysiloxane nanoparticles (FIG. 11). These differences in G′ became more pronounced as the frequency was increased. Nanocomposite hydrogels (b-e) also generally showed an increase in tensile modulus and ultimate tensile strength (UTS) with increased polysiloxane nanoparticles content (Table 3). The degree of hydrogel swelling is directly related to its mechanical properties and many methods to improve mechanical strength are designed to reduce swelling. Thus, the increase in nanocomposite hydrogel G′, tensile modulus, and UTS with increasing amounts of nanoparticles is at least partially attributed to reduced water content at 25° C. For PNIPAAm and other hydrogels, lower water content levels typically also results in a reduction in percent strain at break. However, percent strain at break was observed to generally increase with nanoparticle levels. Higher percent strain at break values coupled with higher tensile modulus and UTS values of nanocomposite hydrogels indicate the enhanced mechanical properties are due in part to the reinforcement of PNIPAAmmatrix by the polysiloxane nanoparticles.

### Example 14

### Volume Phase Transition Temperature (VPTT)

VPTT of swollen hydrogels was determined by differential scanning calorimetry (DSC, TA Instruments Q100).Water-swollen hydrogels were blotted with filter paper and a small piece sealed in a hermetic pan. After cooling to −50° C., the temperature was increased to 50° C. at a rate of 3° C./min for two cycles. The resulting exothermic phase transition peak is characterized by the initial temperature at which the exotherm starts (Tₒ), the peak temperature of the exotherm (Tₘₐₓ) and the enthalpy change (DH) of the phase transition. Data reported are from the second cycle.

PNIPAAm hydrogels exhibit a significant endothermic effect during the volume phase transition due to breaking of hydrogen bonds between water molecules surrounding hydrophobic moieties on the polymer. The VPTT is typically designated by either the onset (Tₒ) or the maximum temperature (Tₘₐₓ) of the endothermic peak. The VPTT and transition enthalpy (DH) values of the nanocomposite hydrogels (b-e) and PNIPAAm control (a) were determined by their respective DSC thermograms of swollen hydrogel specimens (FIG. 10). To values were determined from the intersecting point between two tangent lines from the baseline and slope of the endothermic peak. The VPTT (Tₘₐₓ or Tₒ) of nanocomposite hydrogels (b-e) remained essentially unchanged relative to that of the pure PNIPAAm hydrogel (a). Similarly, the DH values of nanocomposite hydrogels were similar to that of the pure PNIPAAm hydrogel. This indicates that polysiloxane nanoparticles do not interfere with the dissociation of water molecules from hydrophobic groups when heated above the VPTT. The endothermic peak of the nanocomposite hydrogels slightly broadened with increased polysiloxane particle content. Broadening of the endothermic peak of PNIPAAm-based hydrogels is an indicator of more gradual deswelling above the VPTT.

The VPTT of nanocomposite hydrogels with varying levels of nanoparticles (b-e) were compared to that of a pure PNIPAAm hydrogel (a) (i.e., one with no nanoparticles). As illustrated in this example, addition of such polysiloxane nanoparticles may not significantly alter the volume phase transition temperature (VPTT) compared to that of a PNIPAAm hydrogel. This may be desirable (e.g., highly desirable), according to some embodiments, since the VPTT of PNIPAAm hydrogels (˜35° C.) is close to that of body temperature (37° C). Thus, the amount of nanoparticles added may not significantly changing the VPTT. Still, conventional methods may be used to tailor the VPTT of the nanocomposite hydrogel slightly (say to ˜40° C. such that exists in a swollen state in the body) by copolymerizing with a small amount of a second monomer, in some embodiments.

### Example 15

### Preparation of Crosslinked Polysiloxane Collodial Nanoparticles

Octamethylcyclotetrasiloxane (D₄), 1,3,5,7-tetramethyl-1,3,5,7-tetravinylcyclotetrasiloxane (D₄^{Vi}), and silicone oil (trimethylsiloxy terminated PDMS; viscosity=1000 cSt) were purchased from Gelest Inc. Dodecylbenzenesulfonic acid (DBSA, BIO-SOFT® S-101) was received from Stepan Co. Potassium persulfate (K₂S₂O₈), N-isopropylacrylamide (NIPAAm, 97%), 2,2-dimethyl-2-phenyl-acetophenone (DMAP), N-vinylpyrrolidone (NVP) were purchased from Aldrich. N,N0-methylenebisacrylamide (BIS, 99%) was obtained from Acros Organics. 1-[4-(2-Hydroxy)-phenyl]-2-hydroxy-2-methyl-1-propane-1-one (Irgacureφ-2959) was obtained from Ciba. All reagents were used as received. Mouse smooth muscle precursor cells (10T1/2) were obtained from American Type Culture Collection (ATCC).

Polysiloxane colloidal particles were prepared by cationic emulsion polymerization of D₄ and D₄^{Vi}. In a 500-mL water-jacketed polymerization vessel equipped with a mechanical stirrer and Teflon stirring paddle, reflux condenser, and addition funnel, DBSA (0.5 g, 1.53 mmol) was dissolved in deionized (DI) water (200 g). A mixture of D₄ (39.6 g, 133.8 mmol) and D₄^{Vi} (9.9 g, 28.8 mmol) was added dropwise via the addition funnel to the DBSA aqueous solution with constant stirring (300 rpm). The resulting stable emulsion was then heated to 75° C. for 24 h with constant stirring (280 rpm). The final emulsion was cooled, filtered through a 10-mm filter bag, and the pH adjusted to 7 with aqueous NH₄OH (25 wt %). The solid content of the emulsion was determined by weight loss from an aliquot after drying (115° C., 8 h). Emulsion solid content: 17.4% (87% conversion).

Linear copoly(dimethylsiloxane/methylvinylsiloxane) was isolated from the aforementioned colloidal nanoparticles for subsequent characterization. A portion of the final emulsion was precipitated into ethanol, centrifuged, and the isolated clear oil dried under vacuum. ¹H NMR δ (ppm): 0.1 (bs, Si—CH₃), 5.7-6.0 (m, Si—CH═CH₂); ratio of 12:1. Gel permeation chromatography (GPC): Mw/Mn=67,200/36,500 g/mol, PDI=1.84.

These colloidal nanoparticles were subsequently stabilized by crosslinking of the copoly(dimethylsiloxane/methylvinylsiloxane) chains within the nanoparticles via their vinyl groups. The above final emulsion (50 g) was added to a three-neck round bottom (rb) flask equipped with a Teflon-covered stir bar, reflux condenser, and nitrogen (N₂) inlet. After the addition of K₂S₂O₈ (0.5 g), the mixture was reacted at 80° C. for 10 h under N₂. The emulsion was cooled and filtered through a 10-mm filter bag. The resulting colloidal nanoparticles were purified via dialysis (Slide-A-Lyzer® Dialysis Cassette, MWCO=10,000, Pierce Chemical Co.) against daily changes of DI water for 3 days. Emulsion solid content: 8.5%. Dynamic light scattering (DLS): 219 nm (average diameter) and 0.10 (polydispersity, PD) with particles ranging in size from 106 to 531 nm (FIG. 12, upper panel).

Nanocomposite hydrogels were prepared by in situ photopolymerization of aqueous precursor solutions containing NIPAAm monomer, BIS crosslinker, Irgacure-2959 photoinitiator, and crosslinked polysiloxane nanoparticles (FIG. 1). In a 50-mL rb flask equipped with a Teflon-covered stir bar, NIPAAm (1.0 g, 8.84 mmol), BIS (0.02 g, 0. 13 mmol), and Irgacure-2959 (0.08 g, 0.36 mmol) were dissolved in DI water (the total volume equal to 7 mL including the volume of water introduced later by the nanoparticle emulsion) and the solution stirred under N2 for 15 min. Finally, the appropriate amount of emulsion containing crosslinked colloidal nanoparticles was added and the mixture stirred for 10 min under N2. In total, four different hydrogel compositions were prepared with varying amounts of colloidal nanoparticles: (a) pure NIPAAm (no nanoparticles; a control), (b) 0.5 wt %, (c) 1.0 wt %, (d) 1.5 wt %, and (e) 2.0 wt % (wt % solids of nanoparticles with respect to total precursor solution weight).

Hydrogel sheets (1.5 or 0.5 mm thick)were prepared by first pipetting a precursor solution between two clamped glass microscope slides (75×50 mm) separated by polycarbonate spacers of appropriate thickness. The mold was submerged in an ice water bath (w7_C) and exposed to long wave UV light (UV-Transilluminator, 6 mW/cm2, 365 nm) for 30 min. After removal from the mold, the hydrogel sheet was rinsed with DI water and then soaked in DI water for 2 days with daily water changes to remove impurities. Hydrogel sheets (1.5 mm thick) were used to prepare samples for morphological, VPTT, swelling, mechanical, and contact angle studies. Hydrogel sheets (0.5 mm thick) were used for cell-release studies.

For tensile tests, hydrogels were prepared with a “ring” geometry. First, hydrogels were prepared in a hollow tube geometry with a double walled tubular mold composed of an inner glass mandrel (diameter=3 mm) and an outer glass cylinder (diameter=7.9 mm). The tubular mold was filled with a precursor solution and cured while submerged in an ice water bath (˜7° C.) for 30 min under constant rotation such that each surface point of the mold received equal UV intensity and exposure time. The hydrogel tube was removed from the mold and similarly purified as above by rinsing and soaking in DI water. Cutting the resulting hydrogel tube into w3 mm wide pieces afforded hydrogel ring specimens (FIG. 12, upper panel).

Similar crosslinked polysiloxane nanoparticles (ave. diam. −50 nm, with particles from 21-91 nm) were prepared by substituting different surfactants and initiators (FIG. 12, lower panel). Briefly, D₄ (31.2 g) and D₄^{Vi} (7.8 g) were added to a solution of Brij 35 (3.01 g), Brij 78 (6.75 g), and Tergitol NP-40 solution (70%, 5.35 g), in DI water (148.6 g), heated to 80° C. and 10 g of 25 wt % KOH solution added. After 24 h, the emulsion was cooled, filtered, pH adjusted to 7, crosslinked free radically and dialyzed to produce a 22 wt % (83% conversion) emulsion. Smaller particle size may increase the transparency of a corresponding nanoparticle hydrogel. In addition, smaller particle size may enhance mechanical properties and diffusion of target analyte.

Polysiloxane colloidal nanoparticles were prepared by cationic ring-opening emulsion polymerization of D₄ and D₄^{Vi} using DBSA as an inisurf (i.e., an initiator and surfactant) (FIG. 12, upper panel). Linear copoly(dimethylsiloxane/methylvinylsiloxane) (Mw/Mn=67,200/36,500 g/mol) was isolated from the non-crosslinked nanoparticles. Based on ¹H NMR analysis, the ratio of dimethylsiloxane and methyvinylsiloxane repeat units is 4:1. The colloidal nanoparticles were subsequently crosslinked via free radical reaction between the vinyl groups. Surfactant and other reaction impurities were removed from the resultant emulsion via dialysis. This process yielded polysiloxane colloidal nanoparticles having an average diameter of 219 nm (PD ¼ 0.10) with particles ranging in size from 106 to 531 nm.

Nanocomposite hydrogels (b-e) were prepared by photopolymerization aqueous mixtures of NIPAAm monomer, BIS crosslinker, Irgacure-2959 photoinitiator, and crosslinked colloidal polysiloxane nanoparticles ˜7° C. for 30 min (FIG. 12, upper panel). PNIPAAm hydrogels are typically formed by the free radical crosslinking copolymerization of aqueous solutions of NIPAAm and BIS using redox initiators which typically relies inert environments, elevated temperatures, and/or long reaction times [6,63-66]. Thus, photopolymerization was utilized herein to prepare PNIPAAm nanocomposite hydrogels more rapidly with a 30-min cure time. Although NIPAAm may be photocrosslinked in the absence of a crosslinker (e.g. BIS), it may produce elevated sol content [70]. With increasing polysiloxane nanoparticle content, the aqueous precursor solutions became increasingly opaque which may block the transmission of UV light and diminish extent of crosslinking. However, following Soxhlet extraction, hydrogels containing 2 wt % nanoparticles (d) and pure PNIPAAm hydrogel (a) both exhibited no detectable weight loss (<0.1 wt %). Thus, photopolymerization effectively produced nanocomposite hydrogels without significant amounts of non-reacted NIPAAm.

The preparation temperatures (Tprep) at which PNIPAAm hydrogels are formed have been shown to impact their chemical and physical properties by altering hydrogel morphology. PNIPAAm hydrogels formed at Tₚᵣₑₚ<20° C. are morphologically homogeneous whereas those formed at higher temperatures are heterogeneous. At Tprep>w20° C., newly formed insoluble PNIPAAm chains phase separate such that subsequent crosslinking leads to the formation of a macroscopic network of loosely interconnected highly crosslinked polymer rich domains and lightly crosslinked polymer poor domains. As a result, heterogeneous PNIPAAm hydrogels are opaque whereas homogeneous hydrogels are transparent. Heterogeneous PNIPAAm hydrogels display higher swelling ratios but are mechanically weaker compared to the corresponding homogeneous hydrogel. Thus, to optimize mechanical strength of the nanocomposite hydrogels, photopolymerization was conducted at ˜7° C. to obtain nanocomposite hydrogels consisting of a homogeneous PNIPAAm matrix with embedded nanoparticles. The homogeneity of the PNIPAAm hydrogel matrix is confirmed by the optically transparent nature of the pure PNIPAAm hydrogel (a, no nanoparticles). Freeze-drying (i.e., lyophilization) is known to preserve the structure and volume of swollen hydrogels even after all (or almost all) the solvent is removed. SEM micrographs of lyophilized nanocomposite hydrogels revealed that all demonstrated a uniform porous morphology characteristic of homogeneous PNIPAAm hydrogels.

TABLE 1
Composition of the hydrogel materials.

| Hydrogel | Aqueous Precursor Solution |
|---|---|
| (A) PNIPAAm | NIPAAm monomer (12.5 wt %) |
| | BIS crosslinker (2 wt %) |
| | Irgacure photoinitiator (1 wt %) |
| (B) PNIPAAm + | NIPAAm monomer (12.5 wt %) |
| 1 wt % PDMS | BIS crosslinker (2 wt %) |
| | Irgacure photoinitiator (1 wt %) |
| | Polysiloxane colloidal nanoparticles (1 wt %) |
| (C) Porated PNIPAAm + | NIPAAm monomer (12.5 wt %) |
| 1 wt % PDMS | BIS crosslinker (2 wt %) |
| | Irgacure photoinitiator (1 wt %) |
| | Polysiloxane colloidal nanoparticles (1 wt %) |
| | Leachable PEG (MW 1000) (20 wt %) |
| (D) 60% PEG | PEG-DA (MW 575) (60% v/v) |
| | Darocur photoinitiator (1% v/v) |

TABLE 2
Estimated average diffusion rates at temperatures below
and above the volume phase transition temperature.

| | Diffusion | Diffusion |
|---|---|---|
| | Coefficient | Coefficient |
| Material | at 25° C. [m²/s] | at 37° C. [m²/s] |
| (A) PNIPAAm | 8.49 × 10⁻⁷ | 6.37 × 10⁻⁸ |
| (B) PNIPAAm + 1 wt % PDMS | 1.02 × 10⁻⁶ | 7.43 × 10⁻⁸ |
| (C) Porated PNIPAAm + 1 | 1.97 × 10⁻⁶ | 4.25 × 10⁻⁶ |
| wt % PDMS | | |
| (D) 60% PEG | 1.10 × 10⁻⁶ | 1.75 × 10⁻⁶ |

TABLE 3
Tensile properties of nanocomposite hydrogels
(b-e) and pure PNIPAAm hydrogel control (a).

| | Composition | Tensile Properties | | |
|---|---|---|---|---|
| | Solid wt % | Modulus | UTS | % strain at |
| Hydrogel | nanoparticles | (kPa) | (kPa) | break |
| a | 0 | 14.5 ± 1.6 | 6.7 ± 1.5 | 50.4 ± 7.7 |
| b | 0.5 | 16.5 ± 1.0 | 9.8 ± 1.2 | 62.1 ± 3.7 |
| c | 1.0 | 15.6 ± 0.6 | 9.5 ± 0.5 | 64.3 ± 1.9 |
| d | 1.5 | 15.1 ± 2.1 | 10.9 ± 1.5 | 75.1 ± 4.5 |
| e | 2.0 | 17.4 ± 1.5 | 10.8 ± 1.3 | 69.6 ± 3.7 |

What is claimed is:

1. A biosensor membrane comprising a hydrogel comprising cross-linked poly(N-isopropylacrylamide) and one or more polysiloxane colloidal nanoparticles.

2. A biosensor membrane according to claim 1, wherein the biosensor membrane is contiguous.

3. A biosensor membrane according to claim 1, wherein the biosensor membrane comprises one or more pores.

4. A biosensor system comprising a photodiode and a biosensor membrane comprising a hydrogel comprising cross-linked poly(N-isopropylacrylamide) and one or more polysiloxane colloidal nanoparticles.

5. A biosensor system according to claim 4 further comprising a thermocycler and a temperature controller configured and arranged to control the temperature of the thermocycler.

6. A biosensor system according to claim 5 further comprising a display, wherein the biosensor system is configured and arranged in a wrist-watch format.

7. A biosensor system according to claim 5 further comprising an implantable piece comprising at least a portion of the biosensor membrane.

8. A biosensor system according to claim 7, wherein the implantable piece comprises a catheter.

9. A method for cleaning an implanted biosensor membrane comprising providing an implanted biosensor membrane comprising a hydrogel comprising cross-linked poly(N-isopropylacrylamide) and one or more polysiloxane colloidal nanoparticles;
providing a thermocycler in thermal communication with the biosensor membrane; and
cycling the temperature of the thermocycler around the volume phase transition temperature (VPTT) of the biosensor membrane.

10. A method for detecting an analyte comprising
providing an implanted biosensor configured and arranged to detect an analyte, the biosensor comprising a biosensor membrane comprising a hydrogel comprising cross-linked poly(N-isopropylacrylamide) and one or more polysiloxane colloidal nanoparticles;
detecting the analyte.

11. A method according to claim 10, wherein the analyte is selected from the group consisting of cholesterol, a cardiac marker, glucose, and combinations thereof.

12. A method according to claim 10 further comprising:
providing a thermocycler in thermal communication with the biosensor membrane; and
cycling the temperature of the thermocycler around the volume phase transition temperature (VPTT) of the biosensor membrane.

13. A material comprising:
one or more polysiloxane colloidal nanoparticles; and
a hydrogel comprising
a crosslinked poly(N-isopropylacrylamide) or a crosslinked copolymer of N-isopropylacrylamide, and
a monomer selected from the group consisting of acrylic acid, methacrylic acid, acrylamide, and N-vinylpyrrolidone.

14. A biosensor system comprising a photodiode and a biosensor membrane comprising one or more polysiloxane colloidal nanoparticles and a hydrogel comprising cross-linked poly(N-isopropylacrylamide) and a hydrophobic and/or hydrophilic comonomer.

15. A biosensor system according to claim 14 further comprising a thermocycler and a temperature controller configured and arranged to control the temperature of the thermocycler.

16. A biosensor system according to claim 14 further comprising a display, wherein the biosensor system is configured and arranged in a wrist-watch format.

17. A biosensor system according to claim 14 further comprising an implantable piece comprising at least a portion of the biosensor membrane.

18. A biosensor system according to claim 17, wherein the implantable piece comprises a catheter.

19. A method for cleaning an implanted biosensor membrane comprising
providing an implanted biosensor membrane comprising one or more polysiloxane colloidal nanoparticles and a hydrogel comprising cross-linked poly(N-isopropylacrylamide) and a hydrophobic and/or hydrophilic comonomer;
providing a thermocycler in thermal communication with the biosensor membrane; and
cycling the temperature of the thermocycler around the volume phase transition temperature (VPTT) of the biosensor membrane.

20. A method for detecting an analyte comprising
providing an implanted biosensor configured and arranged to detect an analyte, the biosensor comprising a biosensor membrane comprising one or more polysiloxane colloidal nanoparticles and a hydrogel comprising cross-linked poly(N-isopropylacrylamide) and a hydrophobic and/or hydrophilic comonomer; and
detecting the analyte.
